(19) United States
(12) Patent Application Publication
(10) Pub. No.: US 2024/0382283 A1
(43) Pub. Date: Nov. 21, 2024

(54) AUGMENTED REALITY ASSISTANCE FOR OSTEOTOMY AND DISCECTOMY

(71) Applicant: AUGMEDICS LTD., Yokneam Illit (IL)

(72) Inventors: Monica Marie Kuhnert, Chicago, IL (US); Nissan Elimelech, Beerotaim (IL); Stuart Wolf, Yokneam (IL)

(21) Appl. No.: 18/683,680

(22) PCT Filed: Aug. 18, 2022
(86) PCT No.: PCT/IB2022/057736

§ 371 Date: Feb. 14, 2024

Related U.S. Application Data
(60) Provisional application No. 63/281,677, filed on Nov. 21, 2021.
(60) Provisional application No. 63/236,244, filed on Aug. 24, 2021.
(60) Provisional application No. 63/236,241, filed on Aug. 24, 2021.
(60) Provisional application No. 63/234,272, filed on Aug. 18, 2021.

(51) Int. Cl. A61B 90/00; A61B 34/10; A61B 34/20; G06T 7/00; G06T 7/593; G06T 7/62; G06T 19/00

(52) U.S. Cl. CPC A61B 90/37; A61B 34/20; G06T 7/0016; G06T 7/593; G06T 7/62; G06T 19/006; A61B 2034/107; A61B 2090/365; G06T 2200/04; G06T 2207/10081; G06T 2207/20084; G06T 2207/30008; G06T 2210/12; G06T 2210/41

(57) ABSTRACT

Disclosed herein are systems, devices, and methods for image-guided surgery. Some systems include a near-eye unit, having a see-through augmented-reality display, which is configured to display graphical information with respect to a region of interest (ROI) on a body of a patient, including a bone inside the body, that is viewed through the display by a user wearing the near-eye unit. A processor, which is configured to access three-dimensional (3D) image data with respect to the bone, processes the 3D image data so as to identify a first 3D shape of the bone prior to a surgical procedure on the bone and a second 3D shape of the bone following the surgical procedure, to generate, based on the first and second 3D shapes, an image showing a part of the bone that was removed in the surgical procedure, and to present the image on the see-through augmented-reality display.

### CROSS-REFERENCE TO RELATED APPLICATION

This application claims the benefit of U.S. Provisional Patent Application 63/236,241. filed Aug. 24, 2021; U.S. Provisional Patent Application 63/281,677, filed Nov. 21, 2021; U.S. Provisional Patent Application No. 63/234,272, filed Aug. 18, 2021; and U.S. Provisional Patent Application No. 63/236,244, filed Aug. 24, 2021. The entire content of each of these related applications is incorporated herein by reference.

### FIELD

The present disclosure relates to generally to image guided surgery or intervention, and specifically to systems and methods for use of augmented reality in image-guided surgery or intervention and/or to systems and methods for use in surgical computer assisted navigation.

### BACKGROUND

Near-eye display devices and systems can be used in augmented reality systems, for example, for performing image-guided surgery. In this way, a computer-generated image may be presented to a healthcare professional who is performing the procedure such that the image is aligned with an anatomical portion of a patient who is undergoing the procedure. Applicant's own work has demonstrated an image of a tool that is used to perform the procedure can also be incorporated into the image that is presented on the head-mounted display. For example, Applicant's prior systems for image-guided surgery have been effective in tracking the positions of the patient's body and the tool (see, for example, U.S. Pat. No. 9,928,629. U.S. Pat. No. 10,835,296, U.S. Pat. No. 10,939,977, PCT International Publication WO 2019/211741, and U.S. Patent Application publication 2020/0163723.) The disclosures of all these patents and publications are incorporated herein by reference.

### SUMMARY

Embodiments of the present disclosure provide improved systems and methods for presenting augmented-reality near-eye displays. For example, some embodiments of the system assist a surgeon during a medical procedure by displaying the progress of the medical procedure (e.g., bone removal during osteotomy) on the augmented-reality near eye display. By displaying the progress of the medical procedure, the surgeon is able to ensure that the surgery is carried out according to plan, as well as evaluate and verify that the surgery has achieved the desired result. For example, in some embodiments, the system displays or indicates what was completed, i.e., what portion of the bone was already cut and what portion of bone is left to be cut. In some embodiments, the already-cut portion of bone may be indicated on the plan (for example by a different color) or augmented on the image or on reality. This indication may be used to note when a portion of bone was cut in deviation from the plan or only partially cut according to the plan. In some embodiments, tracking of the cutting can be performed based on tool tip tracking or by depth sensing and may be displayed with respect to the plan or even when there is no plan.

In some embodiments, a system for image-guided surgery, comprises a near-eye unit, comprising a see-through augmented-reality display, which is configured to display graphical information with respect to a region of interest (ROI) on a body of a patient, including a bone inside the body, that is viewed through the display by a user wearing the near-eye unit; and a processor, which is configured to access three-dimensional (3D) image data with respect to the bone, to process the 3D image data so as to identify a first 3D shape of the bone prior to a surgical procedure on the bone and a second 3D shape of the bone following the surgical procedure, to generate, based on the first and second 3D shapes, an image showing a part of the bone that was removed in the surgical procedure, and to present the image on the see-through augmented-reality display.

In some embodiments, the processor is configured to access a plan of the surgical procedure and based on the plan, to present a guide for cutting the bone on the see-through augmented-reality display.

In some embodiments, the processor is configured to compare the second 3D shape to the plan and to present an indication of a deviation between the part of the bone that was removed and the plan on the augmented-reality display.

In some embodiments, the processor is configured to present the guide as an outline of an area of the bone that is to be removed, wherein the outline is superimposed on the bone in the see-through augmented-reality display.

In some embodiments, the processor is configured to present on the see-through augmented-reality display an icon indicating a position of a tool used in cutting the bone and a line showing a trajectory that the tool is to take in cutting the bone according to the plan.

In some embodiments, the processor is configured to present the guide on the see-through augmented-reality display together with an image of the bone, such that the guide and the image of the bone are overlaid on an actual location of the bone in the body.

In some embodiments, the processor is configured to present the guide on the see-through augmented-reality display such that the guide is overlaid on actual bone that is to be cut in open surgery.

In some embodiments, the processor is configured to process the 3D image data at one or more times during the surgical procedure so as to identify one or more intermediate 3D shapes of the bone during the surgical procedure, and present the part of the bone removed at each of the one or more times on the see-through augmented-reality display.

In some embodiments, the near-eye unit comprises a depth sensor, which is configured to generate depth data with respect to the ROI, and wherein the processor is configured to generate the image showing the part of the bone using the depth data.

In some embodiments, the processor is configured to measure and display a volume of the bone that was removed during the surgical procedure based on the depth data.

In some embodiments, the processor is configured to process the 3D image data using a convolutional neural network (CNN) so as to generate an indication of a volume of the bone to be removed in a surgical procedure.

In some embodiments, the processor is configured to access 3D tomographic data with respect to the body of the patient and to generate the image showing the part of the bone using the 3D tomographic data.

In some embodiments, a method for image-guided surgery comprises processing first three-dimensional (3D) image data with respect to a bone inside a body of a patient so as to identify a first 3D shape of the bone prior to a surgical procedure on the bone; processing second 3D image data so as to identify a second 3D shape of the bone following the surgical procedure; generating, based on the first and second 3D shapes, an image showing a part of the bone that was removed in the surgical procedure; and presenting the image on a see-through augmented-reality display, such that the image is overlaid on a region of interest (ROI) on the body of a patient that contains the bone inside the body and is viewed through the display.

In some embodiments, the method further comprises accessing a plan of the surgical procedure and based on the plan, presenting a guide for cutting the bone on the see-through augmented-reality display.

In some embodiments, the method further comprises comparing the second 3D shape to the plan and presenting an indication of a deviation between the part of the bone that was removed and the plan on the augmented-reality display.

In some embodiments, presenting the guide comprises displaying an outline of an area of the bone that is to be removed, wherein the outline is superimposed on the bone in the see-through augmented-reality display.

In some embodiments, presenting the guide comprises presenting on the see-through augmented-reality display an icon indicating a position of a tool used in cutting the bone and a line showing a trajectory that the tool is to take in cutting the bone according to the plan.

In some embodiments, presenting the guide comprises displaying the guide on the see-through augmented-reality display together with an image of the bone, such that the guide and the image of the bone are overlaid on an actual location of the bone in the body.

In some embodiments, presenting the guide comprises displaying the guide on the see-through augmented-reality display such that the guide is overlaid on actual bone that is to be cut in open surgery.

In some embodiments, the method further comprises acquiring and processing further 3D image data at one or more times during the surgical procedure so as to identify one or more intermediate 3D shapes of the bone during the surgical procedure, and presenting the part of the bone removed at each of the one or more times on the see-through augmented-reality display.

In some embodiments, the 3D image data comprise depth data, which are acquired with respect to the ROI by a depth sensor, and wherein processing the first and second 3D image data comprises generating the image showing the part of the bone using the depth data.

In some embodiments, the method further comprises measuring and displaying a volume of the bone that was removed during the surgical procedure based on the depth data.

In some embodiments, processing the first 3D image data uses a convolutional neural network (CNN) so as to generate an indication of a volume of the bone to be removed in a surgical procedure.

In some embodiments, processing the first 3D image data comprises accessing 3D tomographic data with respect to the body of the patient and generating the image showing the part of the bone using the 3D tomographic data.

In some embodiments, a computer software product, comprising a tangible, non-transitory computer-readable medium in which program instructions are stored, which instructions, when read by a computer, cause the computer to process first three-dimensional (3D) image data with respect to a bone inside a body of a patient so as to identify a first 3D shape of the bone prior to a surgical procedure on the bone, to process second 3D image data so as to identify a second 3D shape of the bone following the surgical procedure, to generate, based on the first and second 3D shapes, an image showing a part of the bone that was removed in the surgical procedure, and to present the image on a see-through augmented-reality display, such that the image is overlaid on a region of interest (ROI) on the body of a patient that contains the bone inside the body and is viewed through the display.

In some embodiments, a system for image-guided surgery comprises a see-through augmented-reality display configured to display a bone with respect to a region of interest (ROI) on a body of a patient, the bone being disposed inside the patient; and a processor and a memory for storing instructions that, when executed by the processor cause the system to: access three-dimensional (3D) image data related to the bone; determine a first 3D shape of the bone prior to removing a portion of the bone; determine a second 3D shape of the bone after the portion is removed; generate an image of the portion of the bone removed; and displaying the image on the see-through augmented-reality display so as to be viewable by the user in the ROI on the body of the patient.

In some embodiments, a method for image-guided surgery comprises determining a first three-dimensional (3D) shape of a bone prior to removing a portion of the bone, the bone being disposed inside a patient; determining a second 3D shape of the bone after the portion is removed; generating, based on the first and second 3D shapes, an image of the portion of the bone removed; and displaying the image on a see-through augmented-reality display so as to be viewable by a user in a region of interest (ROI) on the body of the patient.

In some embodiments, the method further comprises displaying a plan for the removal of the portion of the bone on the see-through augmented-reality display, the plan illustrating the bone after one or more cuts to the bone; and if there is a deviation between the second 3D shape and the plan, displaying an indication of the deviation on the see-through augmented-reality display.

For purposes of summarizing the disclosure, certain aspects, advantages, and novel features are discussed herein. It is to be understood that not necessarily all such aspects, advantages, or features will be embodied in any particular embodiment of the disclosure, and an artisan would recognize from the disclosure herein a myriad of combinations of such aspects, advantages, or features.

The present disclosure will be more fully understood from the following detailed description of the embodiments thereof, taken together with the drawings in which:

### BRIEF DESCRIPTION OF THE DRAWINGS

Non-limiting features of some embodiments of the invention are set forth with particularity in the claims that follow. The following drawings are for illustrative purposes only and show non-limiting embodiments. Features from different figures may be combined in several embodiments.

FIG. 1 is a schematic pictorial illustration showing a system for image-guided surgery, in accordance with an embodiment of the disclosure;

FIG. 2A is a schematic pictorial illustration showing details of a near-eye unit that is used for image-guided surgery, in accordance with an embodiment of the disclosure;

FIG. 2B is a schematic pictorial illustration showing details of a head-mounted unit that is used for image-guided surgery, in accordance with another embodiment of the disclosure;

FIG. 3 is a flow chart that schematically illustrates a method for image-guided surgery, in accordance with an additional embodiment of the disclosure;

FIGS. 4A, 4B and 4C are schematic representations of a display showing CT images, which can be used by a surgeon in planning an osteotomy, in accordance with an embodiment of the disclosure;

FIG. 5 is a schematic representation of a display showing images presented during surgery, in accordance with an embodiment of the disclosure; FIG. 6 is a schematic representation of a display showing a CT image which can be used by a surgeon in planning an osteotomy, in accordance with another embodiment of the disclosure; and

FIG. 7 is a schematic pictorial view of a part of a patient's back that is viewed through an augmented-reality display during osteotomy, in accordance with an embodiment of the disclosure.

### DETAILED DESCRIPTION OF EMBODIMENTS

### Overview

Osteotomy (bone cutting), for example of the spine, may be performed for various reasons, including decompression, correction, and access for interbodies. Decompression, for example, is commonly used to free nerves from the bone and thus eliminate pain resulting from pressure on the nerves. Once the nerve has been decompressed, an interbody is sometimes placed between two vertebrae to increase the disc space and keep pressure off the nerve. Osteotomy may be performed, for example, by drilling at multiple points in the bone to a certain depth.

Discectomy is the surgical removal of abnormal disc material that presses on a nerve or on the spinal cord. The procedure involves removing a portion of an intervertebral disc. A laminotomy is often performed in conjunction with the discectomy to remove a part of the vertebra (the lamina), and thus provide access to the intervertebral disc.

Embodiments of the present disclosure that are described herein provide systems, methods and software for image-guided surgery that assist in the performance of medical procedures, for example, osteotomies or discectomies. Some embodiments assist the surgeon in ensuring that the surgery is carried out according to plan, as well as in evaluating and verifying that the surgery has achieved the desired result.

Some embodiments of the disclosed systems include a near-eye unit, comprising a see-through augmented-reality (AR) display, which displays graphical information with respect to a region of interest (ROI) on a body of a patient. The near-eye unit may have the form, for example. of spectacles or a head-up display mounted on suitable headwear. In some embodiments, the ROI includes a bone inside the body, which is viewed through the display by a user, such as a surgeon, wearing the near-eye unit. In some embodiments, a processor accesses 3D image data with respect to the bone and processes the 3D image data so as to identify the 3D shapes of the bone prior to a surgical procedure on the bone, during the surgical procedure on the bone, and following the surgical procedure on the bone. (The surgical procedure on the bone may be a part of a larger and more complex procedure, such as discectomy, which includes other steps in addition to cutting the bone.) In some embodiments based on the 3D shapes, the processor generates and presents an image on the see-through AR display showing a part of the bone that was removed in the surgical procedure. In some embodiments, the processor accesses a plan of the surgical procedure and based on the plan, presents a guide on the see-through AR display for cutting the bone. In some embodiments, the guide may include an indication of portions of the bone that have already been removed.

In some embodiments, the near-eye unit comprises a depth sensor, which generates depth data with respect to the ROI. In some embodiments, the processor generates the image showing the part of the bone using the depth data. Additionally or alternatively, the processor accesses 3D tomographic data, such as CT images, with respect to the body of the patient and generates the image using the 3D tomographic data.

The capabilities of the systems described herein may be applied at several stages in a medical procedure (e.g., osteotomy or discectomy):

1. Planning—Prior to the procedure, in some embodiments, the surgeon can use planning software to plan the bone cut. In some embodiments, the planning of the bone cut can be performed by indicating the portion of the bone to be cut on preoperative scans, such as a 3D CT or MRI scan or a 2D scan, such as a fluoroscopic scan, or a combination of two or more of such scans. The planning may be performed on 2D planar views of the volume and/or on a 3D view. The planned cut may be in the form of a line on the bone surface, points on the bone surface, a plane through the bone, a 3D region or a volume of the bone, a surface of such a region or volume, or other forms. The bone cut may be planned in two dimensions and/or three-dimensions. Optionally, the ROI may be displayed on the AR display with the portion of the bone that is to be cut already removed, thus virtually demonstrating the end result in order to assist the user in planning the bone cut.
2. Cutting Navigation—Optionally, during the procedure, an intraoperative scan is performed, for example, a 2D and/or 3D scan. The preoperative scan and the intraoperative scan may then be registered one with the other, and the plan, for example in the form of a bone cut contour or other indication, may be presented on the intraoperative scan in the AR display, based on the registration. Alternatively or additionally, the plan may be displayed on the preoperative scan, which may be updated using real-time depth data measured by a depth sensor and displayed, for example on the near-eye unit. In some embodiments, during the procedure, the system displays a virtual guide for a bone cutting tool to assist the surgeon in navigating the tool along or within the planned lines, points, surface or volume. In some embodiments, the 3D cutting plan can be overlaid on reality (without displaying patient image data), optionally in a semi-or partially-transparent manner, in alignment with and oriented according to the patient anatomy. This plan may be used as a guide for cutting.

In some embodiments, as the surgeon cuts the bone, the outline or indication of the plane of bone to be removed according to the plan changes depending on the cutting tool tip location, for example based on depth of penetration. The plan outline or plane can be displayed from a point of view defined by the tool orientation, based on tool tracking. This mode may be compatible with, for example, a “Tip View” mode, in which the patient spine model, which can be generated based on a CT scan and presented on the near-eye display, changes according to the tool tip location. In the Tip View mode, in some embodiments, the upper surface of the patient spine model is defined by the tip location and the tool orientation, for example orthogonal to the tool trajectory or orientation, and tip location. In this mode, the patient spine model is “cut” up to that surface and only a portion of the model is displayed.

Alternatively or additionally, the image or patient spine model of the ROI that is presented on the AR display changes dynamically according to the cutting performed and based on tracking of the cutting as described above. Thus, if a drill is used, for example, holes may be formed in the model correspondingly. This dynamic update may be performed during the procedure and/or during cutting, such that at the end of the cutting, the surgeon is presented with a model showing the entire bone portion removed.

Further alternatively or additionally, a virtual volume according to the plan may be displayed to the user separately, rather than overlaid on the ROI, and may be updated by tracking the cutting that has been performed.

The above features make it possible to display or indicate what was done already, i.e., what portion of the bone was already cut and what portion of bone is left to be cut. According to some aspects, the already-cut portion may be indicated on the plan (for example by a different color) or augmented on the image or on reality. This indication may be used to note when a portion of bone was cut in deviation from the plan or only partially according to plan. Tracking of the cutting may be performed based on tool tip tracking or by depth sensing and may be displayed with respect to a plan or even when there is no plan.

3. Post-cutting—In some embodiments, once the surgeon has removed the bone, it can be shown on the AR display by segmentally removing the indicated portion of the bone from the displayed scan, for example by rendering it transparent. The surgeon may then review the anatomy of the patient in the ROI without the bone, including anatomical portions that were previously obscured or concealed by the removed bone portion.

In some embodiments, to track cutting using depth sensing, a first depth image of the bone is captured prior to cutting. During the cutting, additional depth images are captured. The capturing may be performed upon user request or automatically, either continuously or at predefined time intervals or events. In some embodiments, each depth image is compared to the previous one, and the system identifies whether a bone portion was removed or not, i.e., whether cutting was performed. When a difference in the volume of the bone is identified relative to a previous depth image, the difference, indicating the portion of bone that was removed, may be displayed and compared to the plan. The display enables the user to visualize the size and shape of the bone portion that was removed. The depth camera may be calibrated relative to a tool tracking system used in the surgery. Alternatively, or additionally, the depth camera images may be registered with the CT model, for example using feature matching. This calibration and registration may allow comparison between successive depth camera images and between the depth camera images and the CT model and the plan.

Optionally, the actual bone portion that was removed may be scanned using a depth sensor, which may be integrated with the near-eye unit, as noted above. Accordingly, the processor generates a 3D model of the removed bone portion and indicates the actual removed bone portion on the scan. The processor may display or otherwise indicate the portion of the bone that has actually been removed in comparison with the plan, for example by outlining the planned and removed portions. Alternatively or additionally, the processor may display a scan of the ROI anatomy without the actual removed bone portion for comparison with the plan. The surgeon may use the displayed information to verify that the procedure was performed properly, to correct the cut as necessary, and/or to perform any other necessary operations.

Optionally, if the removed portion of the bone is to be replaced by an implant, the surgeon may use the model of the removed bone portion to select a suitable implant. Additionally or alternatively, depth sensing may be used to model a desired implant and/or compare or match the implant to the removed bone portion. Alternatively, a predefined model or dimensions of the implant may be used.

According to another embodiment, a tracking sensor, mounted on the near-eye unit, for example, may track the cutting tool, such as a drill, to identify the path of the actual cut performed by the surgeon, based on the dimensions and trajectory of the tool. The processor may then present to the surgeon the actual cut performed. Additionally or alternatively, the processor may present the planned cut path on the AR display during the cutting process. If depth sensing is used, a model of the portion of the bone that has been cut may be aligned with the actual cut path. All the above information may be used by the surgeon to confirm, change, correct, or perform any other necessary operation.

According to another embodiment, a preoperative MRI scan may be registered with a preoperative CT scan to display both the bone and soft tissue in the ROI. Thus, when the planned and/or actual cut bone portion is removed from the displayed anatomical scan of the ROI, the surgeon is also able to see the soft tissue located beneath the removed bone portion.

### SYSTEM DESCRIPTION

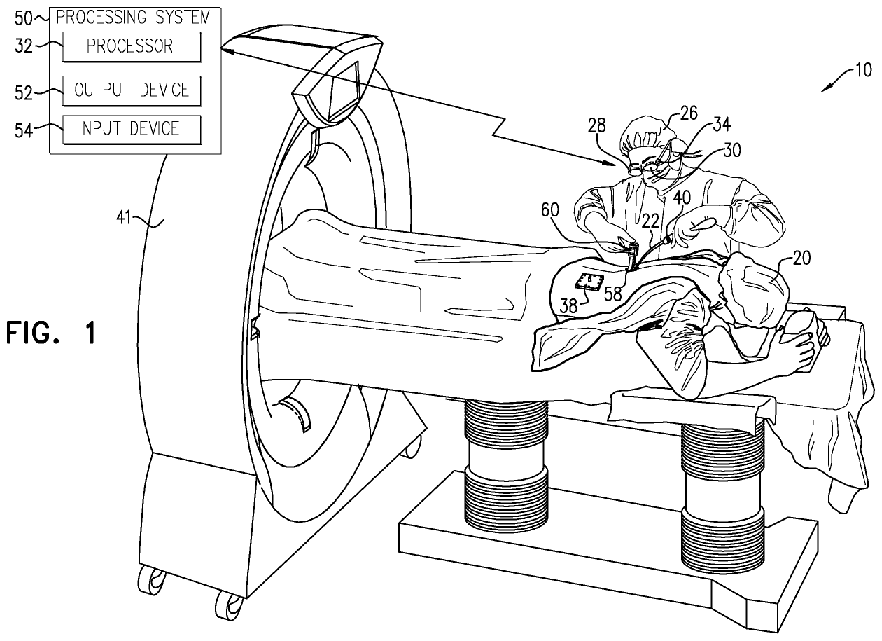

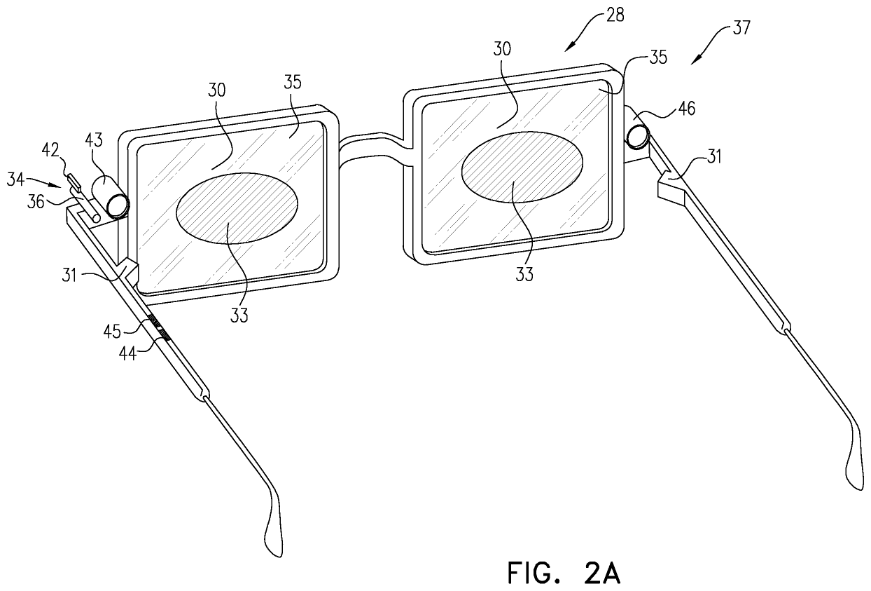

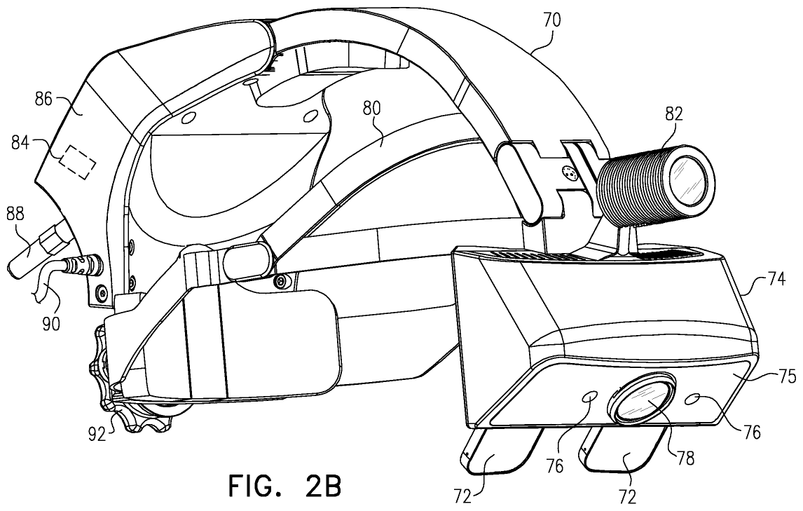

Reference is now made to FIGS. 1 and 2A, which schematically illustrate an exemplary system 10 for image-guided surgery, in accordance with some embodiments of the disclosure. For example, FIG. 1 is a pictorial illustration of the system 10 as a whole, while FIG. 2A is a pictorial illustration of a near-eye unit that is used in the system 10. The near eye unit illustrated in FIGS. 1 and 2A is configured as a head-mounted unit 28 In some embodiments, the near-eye unit can be configured as a head-mounted AR display (HMD) unit 70 and described hereinbelow. In FIG. 1. the system 10 is applied in a medical procedure on a patient 20 using image-guided surgery. In this procedure, a tool 22 is inserted via an incision in the patient's back in order to perform a surgical intervention. Alternatively, the system 10 and the techniques described herein may be used, mutatis mutandis, in other surgical procedures.

Methods for optical depth mapping can generate a three-dimensional (3D) profile of the surface of a scene by processing optical radiation reflected from the scene. In the context of the present description and in the claims, the terms depth map, 3D profile, and 3D image are used interchangeably to refer to an electronic image in which the pixels contain values of depth or distance from a reference point, instead of or in addition to values of optical intensity.

In some embodiments, depth mapping systems can use structured light techniques in which a known pattern of illumination is projected onto the scene. Depth can be calculated based on the deformation of the pattern in an image of the scene. In some embodiments, depth mapping systems use stereoscopic techniques, in which the parallax shift between two images captured at different locations is used to measure depth. In some embodiments, depth mapping systems can sense the times of flight of photons to and from points in the scene in order to measure the depth coordinates. In some embodiments, depth mapping systems control illumination and/or focus and can use various sorts of image processing techniques.

In the embodiment illustrated in FIG. 1, a user of the system 10, such as a healthcare professional 26 (for example a surgeon performing the procedure), wears the near-eye unit 28. In some embodiments, the near-eye unit 28 includes one or more see-through displays 30, for example as described in the above-mentioned U.S. Pat. No. 9,928,629 or in the other patents and applications cited above.

In some embodiments, the one or more see-through displays 30 include an optical combiner. In some embodiments, the optical combiner is controlled by one or more processors 32. In some embodiments, the one or more processors 32 is disposed in a central processing system 50. In some embodiments, the one or more processors 32 is disposed in the head-mounted unit 28. In some embodiments, the one or more processors 32 are disposed in both the central processing system 50 and the head-mounted unit 28 and can share processing tasks and/or allocate processing tasks between the one or more processors 32.

In some embodiments, the one or more see-through displays 30 display an augmented-reality image to the healthcare professional 26. In some embodiments, the augmented reality image viewable through the one or more see-through displays 30 is a combination of objects visible in the real world with the computer-generated image. In some embodiments, each of the one or more see-through displays 30 comprises a first portion 33 and a second portion 35. In some embodiments, the one or more see-through displays 30 display the augmented-reality image such that the computer-generated image is projected onto the first portion 33 in alignment with the anatomy of the body of the patient 20 that is visible to the healthcare professional 26 through the second portion 35.

The alignment of this image with the patient's anatomy can be achieved by means of a registration process, which utilizes a registration marker mounted on an anchoring implement, for example a clamp marker 60 attached to a clamp 58 or pin. For this purpose, an intraoperative CT scan of the ROI may be performed, including the registration marker, in which an image is captured of the ROI and registration marker using the tracking system. The two images are then registered based on the registration marker.

In some embodiments, the computer-generated image includes a virtual image of one or more tools 22. In some embodiments, the system 10 combines at least a portion of the virtual image of the one or more tools 22 into the computer-generated image. For example, some or all of the tool 22 may not be visible to the healthcare professional 26 because, for example, a portion of the tool 22 is hidden by the patient's anatomy (e.g., a distal end of the tool 22). In some embodiments, the system 10 can display the virtual image of at least the hidden portion of the tool 22 as part of the computer-generated image displayed in the first portion 33. In this way, the virtual image of the hidden portion of the tool 22 is displayed on the patient's anatomy. In some embodiments, the portion of the tool 22 hidden by the patient's anatomy increase and/or decreases over time or during the procedure. In some embodiments, the system 10 increase and/or decreases the portion of the tool 22 included in the computer-generated image based on the changes in the portion of the tool 22 hidden by the patient's anatomy over time. According to some aspects, the image presented on the one or more see-through displays 30 is aligned with the body of the patient 20. According to some aspects, misalignment of the image presented on the one or more see-through displays 30 with the body of the patient 20 may be allowed. In some embodiments, the misalignment may be 0-1 mm, 1-2 mm, 2-3 mm, 3-4 mm, 4-5 mm, 5-6 mm, and overlapping ranges therein. According to some aspects, the misalignment may typically not be more than about 5 mm. In order to account for such a limit on the misalignment of the patient's anatomy with the presented images, the position of the patient's body, or a portion thereof, with respect to the head-mounted unit 28 can be tracked. For example, in some embodiments, a patient marker 38 and/or the bone marker 60 attached to an anchoring implement or device such as a clamp 58 or pin, for example, may be used for this purpose, as described further hereinbelow.

When an image of the tool 22 is incorporated into the computer-generated image that is displayed on the head-mounted unit 28, the position of the tool 22 with respect to the patient's anatomy should be accurately reflected. For this purpose, the position of the tool 22 or a portion thereof, such as the tool marker 40, is tracked by the system 10. In some embodiments, the system 10 determines the location of the tool 22 with respect to the patient's body such that errors in the determined location of the tool 22 with respect to the patient's body are reduced. For example, in certain embodiments, the errors may be 0-1 mm. 1-2 mm. 2-3 mm, 3-4 mm, 4-5 mm, and overlapping ranges therein.

In some embodiments, the near-eye unit 28 includes a tracking sensor 34 to facilitate determination of the location and orientation of the near-eye unit 28 with respect to the patient's body and/or with respect to the tool 22. In some embodiments, the tracking sensor 34 can also be used in finding the position and orientation of the tool 22 with respect to the patient's body. In one embodiment, the tracking sensor 34 comprises an image-capturing device 36, such as a camera, which captures images of the patient marker 38, the clamp marker 60, and/or the tool marker 40. For some applications, an inertial-measurement unit 44 is also disposed on the near-eye unit to sense movement of the user's head.

In some embodiments, the tracking sensor 34 includes a light source 42. In some embodiments, the light source 42 is mounted on the head-mounted unit 28. In some embodiments, the light source 42 irradiates the field of view of the image-capturing device 36 such that light reflects from the patient marker 38, the bone marker 60, and/or the tool marker 40 toward the image-capturing device 36. In some embodiments, the image-capturing device 36 comprises a monochrome camera with a filter that passes only light in the wavelength band of light source 42. For example, the light source 42 may be an infrared light source, and the camera may include a corresponding infrared filter. In some embodiments, the patient marker 38, the bone marker 60, and/or the tool marker 40 comprise patterns that enable a processor to compute their respective positions, i.e., their locations and their angular orientations, based on the appearance of the patterns in images captured by the image-capturing device 36. Suitable designs of these markers and methods for computing their positions and orientations are described in the patents and patent applications incorporated herein and cited above.

In addition to or instead of the tracking sensor 34, the head-mounted unit 28 can include a depth sensor 37. In the embodiment shown in FIG. 2A, the depth sensor 37 comprises a light source 46 and a camera 43. In some embodiments, the light source 46 projects a pattern of structured light onto the region of interest (ROI) that is viewed through the one or more displays 30 by a user, such as professional 26, who is wearing the head-mounted unit 28. The camera 43 can capture an image of the pattern on the ROI and output the resulting depth data to the processor 32 and/or processor 45. The depth data may comprise, for example, either raw image data or disparity values indicating the distortion of the pattern due to the varying depth of the ROI. In some embodiments, the processor 32 computes a depth map of the ROI based on the depth data generated by the camera 43.

In some embodiments, the camera 43 also captures and outputs image data with respect to the markers in system 10, such as patient marker 38, bone marker 60, and/or tool marker 40. In this case, the camera 43 may also serve as a part of tracking sensor 34, and a separate image-capturing device 36 may not be needed. For example, the processor 32 may identify patient marker 38, bone marker 60, and/or tool marker 40 in the images captured by camera 43. The processor 32 may also find the 3D coordinates of the markers in the depth map of the ROI. Based on these 3D coordinates, the processor 32 is able to calculate the relative positions of the markers, for example in finding the position of the tool 22 relative to the body of the patient 20, and can use this information in generating and updating the images presented on head-mounted unit 28.

In some embodiments, the depth sensor 37 may apply other depth mapping technologies in generating the depth data. For example, the light source 46 may output pulsed or time-modulated light, and the camera 43 may be modified or replaced by a time-sensitive detector or detector array to measure the time of flight of the light to and from points in the ROI. As another option, the light source 46 may be replaced by another camera, and the processor 32 may compare the resulting images to those captured by the camera 43 in order to perform stereoscopic depth mapping. These and all other suitable alternative depth mapping technologies are considered to be within the scope of the present disclosure.

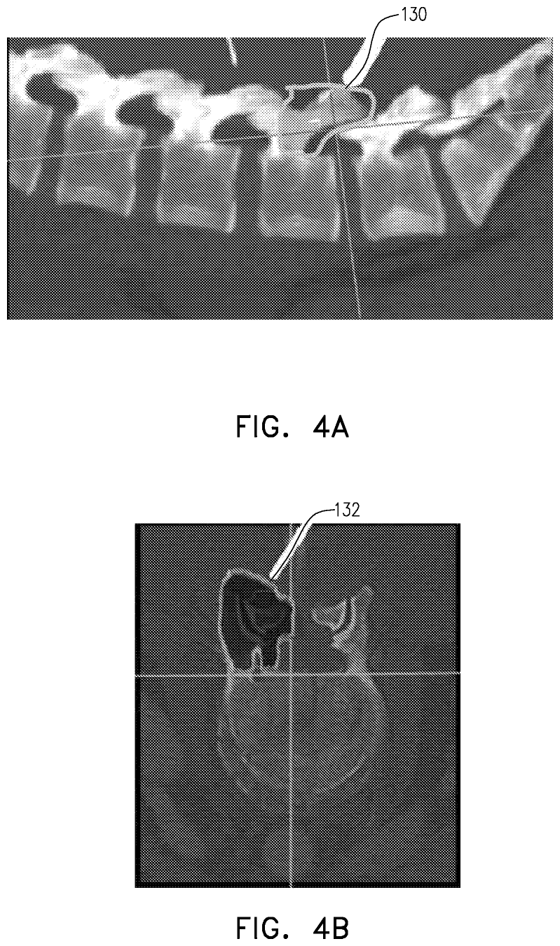

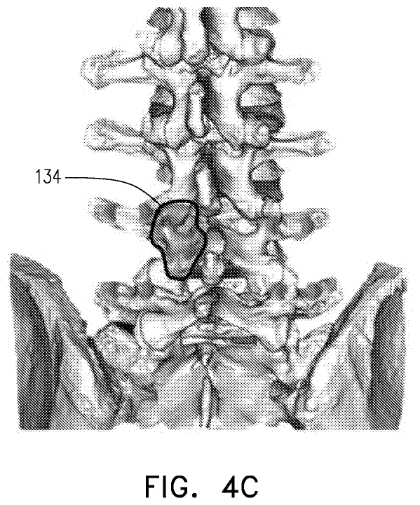

In the pictured embodiment, system 10 also includes a tomographic imaging device, such as an intraoperative computerized tomography (CT) scanner 41. Alternatively or additionally, processing system 50 may access or otherwise receive tomographic data from other sources; and the CT scanner itself is not an essential part of the present system. In some embodiments, regardless of the source of the tomographic data, the processor 32 can computes a transformation over the ROI so as to register the tomographic images with the depth maps that it computes on the basis of the depth data provided by depth sensor 37. The processor 32 can then apply this transformation in presenting a part of the tomographic image on the one or more displays 30 in registration with the ROI viewed through the one or more displays 30. This functionality is described further hereinbelow with reference to FIG. 4A.

In some embodiments, in order to generate and present an augmented reality image on the one or more displays 30, the processor 32 computes the location and orientation of the head-mounted unit 28 with respect to a portion of the body of patient 20, such as the patient's back. In some embodiments, the processor 32 also computes the location and orientation of the tool 22 with respect to the patient's body. In some embodiments, the processor 45, which can be integrated within the head-mounted unit 28, may perform these functions. Alternatively or additionally, the processor 32, which is disposed externally to the head-mounted unit 28 and can be in wireless communication with the head-mounted unit 28, may be used to perform these functions. The processor 32 can be part of the processing system 50, which can include an output device 52, for example a display, such as a monitor, for outputting information to an operator of the system, and/or an input device 54, such as a pointing device, a keyboard, or a mouse, to allow the operator to input data into the system.

In general, in the context of the present description, when a computer processor is described as performing certain steps, these steps may be performed by external computer processor 32 and/or by computer processor 45, which is integrated within the near-eye unit. The processor or processors carry out the described functionality under the control of suitable software, which may be downloaded to system 10 in electronic form, for example over a network, and/or stored on tangible, non-transitory computer-readable media, such as electronic, magnetic, or optical memory.

FIG. 2B is a schematic pictorial illustration showing details of a head-mounted AR display (HMD) unit 70, according to another embodiment of the disclosure. HMD unit 70 may be worn by the healthcare professional 26, and may be used in place of the head-mounted unit 28 (FIG. 1). HMD unit 70 comprises an optics housing 74 which incorporates a camera 78, and in the specific embodiment shown, an infrared camera. In some embodiments, the housing 74 comprises an infrared-transparent window 75, and within the housing, i.e., behind the window, are mounted one or more, for example two, infrared projectors 76. One of the infrared projectors and the camera may be used, for example, in implementing a pattern-based depth sensor.

In some embodiments, mounted on housing 74 are a pair of augmented reality displays 72, which allow professional 26 to view entities, such as part or all of patient 20, through the displays, and which are also configured to present to surgeon 22 images or any other information. In some embodiments, the displays 72 present planning and guidance information, as described above.

In some embodiments, the HMD unit 70 includes a processor 84, mounted in a processor housing 86, which operates elements of the HMD unit. In some embodiments, an antenna 88, may be used for communication, for example with processor 52 (FIG. 1).

In some embodiments, a flashlight 82 may be mounted on the front of HMD unit 70. In some embodiments, the flashlight may project visible light onto objects so that professional is able to clearly see the objects through displays 72. In some embodiments, elements of the HMD unit 70 are powered by a battery (not shown in the figure), which supplies power to the elements via a battery cable input 90.

In some embodiments, the HMD unit 70 is held in place on the head of professional 26 by a head strap 80, and the professional may adjust the head strap by an adjustment knob 92.

### Methods for Planning and Execution of Surgery

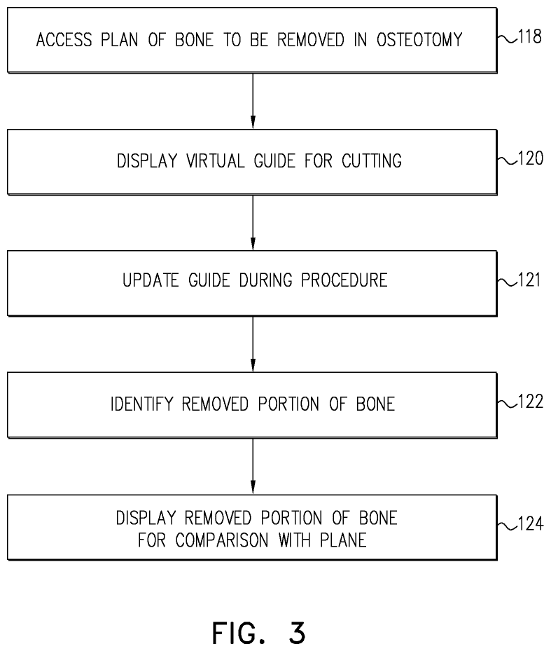

FIG. 3 is a flow chart that schematically illustrates a method for image-guided surgery, in accordance with an additional embodiment of the disclosure. This method is applicable particular in osteotomies, to assist in visualizing the portion of a bone that is removed in the procedure. In such a procedure, in some embodiments, the processing system 50 accesses a plan to remove at least a portion of a bone at planning step 118. In some embodiments, the plan is made by the surgeon. In some embodiments, the plan is to remove a certain volume of the bone in question. such as a part of one or more vertebrae. In some embodiments, the present method can assist the surgeon in implementing the plan precisely and in comparing the volume of the bone that has been cut out to the planned volume. This method may similarly be applied, mutatis mutandis, in other sorts of surgical procedures, such as discectomies.

For the purpose of planning in some embodiments, prior to cutting of the bone, the processor 32 may process depth data generated by the depth sensor 37. In some embodiments, the processor 32 identifies the 3D shape of the bone, for example, by generating a point cloud. Additionally or alternatively, the processor may use previously acquired 3D data, such as a preoperative CT scan, in identifying the 3D shape of the bone. In some embodiments, the processor 32 then superimposes the planned cut from step 118 on the 3D shape in order to generate and display a virtual guide on the one or more displays 30, at a guide presentation step 120. In some embodiments, the 3D cutting plan can be overlaid on reality (without displaying patient image data), optionally in a semi-or partially-transparent manner. In some embodiments, the top plane of the plan is overlaid on the patient and is aligned with and oriented according to the patient anatomy. The plan may be used as a guide for cutting. Examples of images that can be presented as part of the process of steps 118 and 120 are shown in the figures that follow.

In some embodiments, as the surgeon cuts the bone, the outline or indication of the plane of bone to be removed according to the plan changes, for example according to the cutting tool tip location, including depth of tool penetration, and the display is modified accordingly, at a guide update step 121. In some embodiments, the plan outline or plane is displayed from a point of view defined by the tool orientation and based on tool tracking. This mode is especially compatible with a “Tip View” mode, in which the patient spine model, which is generated based on the CT scan, is displayed on the near-eye display. In some embodiments, the view of the model changes according to the tool tip location, such that the upper surface of the model is the upper plane defined by the tool orientation and tip location. For example, the model may be “cut” up to that surface such that only a portion of the model is displayed.

Alternatively or additionally, at a step 122, a removed portion of the bone is identified. In some embodiments, the removed portion of the bone is identified by tracking the tip of the cutting tool or by using depth sensing technologies. In some embodiments, the image or model of the ROI that is presented on the AR display, such as a patient spine model, may be changed dynamically according to the cutting performed and based on tracking of the cutting as described above. Thus, if a drill is used, for example, holes may be formed in the model correspondingly. This dynamic update may be performed during the procedure and during cutting, and allows the surgeon to follow or track the cutting operation and reevaluate, if necessary. In some embodiments, at the end of the cutting, the surgeon is presented with a model showing the entire bone portion removed. According to some aspects, the bone portions removed during the cutting procedure may be dynamically compared to the cutting plan.

Further alternatively or additionally, a virtual volume according to the plan may be displayed to the user (not overlaid on the ROI) and updated by tracking the cutting that has been performed.

In some embodiments and for the purpose of comparison of the plan to execution after the entire bone portion has been cut, processor 32 can access and process new depth data in order to identify the modified 3D shape of the bone. Based on the difference between the 3D shapes, the processor 32 identifies the portion of the bone that was removed. In some embodiments, the processor 32 can then display an image showing the part of the bone that was removed in the surgical procedure, at an excision identification step 122 and a display step 124. The surgeon can compare this image to the plan in order to verify that the osteotomy was completed according to the plan. In some embodiments, the processor 32 can display both images, i.e., of the removed bone volume and of the planned volume, simultaneously to facilitate comparison between the two. In some embodiments, the images may be displayed in an adjacent manner, one on top of the other (for example superimposed as in an augmented reality display), or in other display modes.

Hence, steps 122 and 124 may be performed iteratively during the procedure at one or more stages of the cutting procedure. Additionally or alternatively, these steps may be performed at the end of the cutting procedure, when the entire bone portion to be cut has been removed. During the cutting procedure, it is thus possible to display or indicate what was done already, i.e., what portion of the bone was already cut and what portion of bone is left to be cut. The already-cut portion may be indicated on the plan, for example by marking it in a different color, or augmented on the image or on reality. This display may indicate that a part of the bone was cut in deviation from the plan or only partially according to plan.

Alternatively, the processor 32 may display to the surgeon only the removed portion of the bone, without comparison to the plan. The processor 32 may thus demonstrate the removed volume and assist in confirming the procedure or in deciding whether a correction or a further operation is required, for example. Additionally or alternatively, in cases in which an implant is to be placed in the body in the area of the removed portion of the bone, the surgeon and/or processor 32 may use the model of the removed bone portion to select a suitable implant or to determine whether a particular implant is suitable. On this basis, a suitable implant may be selected from a database, for example. When comparing the removed bone volume to a specific implant, size data may be provided with respect to the implant, or it may be generated using the depth sensing techniques described above.

As explained above, the method of FIG. 3 includes functions associated both with guiding the surgical procedure (steps 120 and 121) and evaluating and displaying the results of the procedure (steps 122 and 124). These functions can advantageously be used in combination, as described above. Alternatively, the guiding functions may be carried out without relation to the evaluation and display functions, and the evaluation and display functions may similarly be carried out independently of any prior guidance.

FIGS. 4A-4C are CT images, which can be used by a surgeon in the process of planning an osteotomy, in accordance with an embodiment of the disclosure. In this embodiment, a part of one of the vertebrae is to be removed in a minimally-invasive procedure. FIG. 4A is a sagittal slice of an area of the spine on which the surgery is to take place, while FIG. 4B is a transverse slice of the same area. In some embodiments, the processor 32 presents these slices on a display and enables the surgeon to scroll the slices through the planes of interest. In some embodiments, in each plane, the surgeon marks relevant areas 130, 132 of the bone that is to be removed. The processor 32 uses the marked areas in identifying a 3D volume 134 of the bone that is to be removed as is illustrated in FIG. 4C.

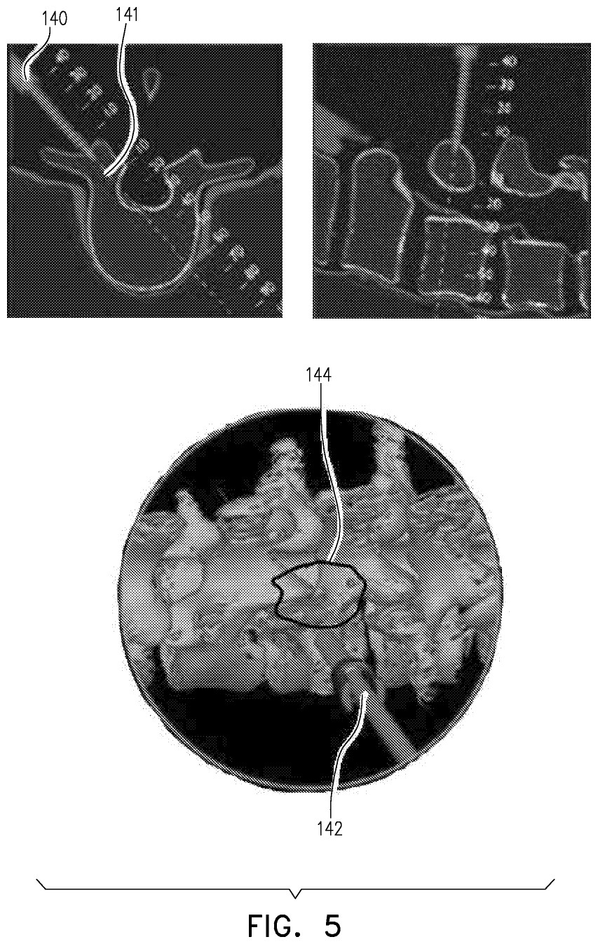

FIG. 5 shows images presented by the processing system 50 during the surgery, to guide the surgeon in cutting the bone according to the plan of FIGS. 4A-4C, in accordance with an embodiment of the disclosure. In some embodiments, the images may be overlaid on the surgeon's field of view on the one or more displays 30. Alternatively or additionally, the images may be presented on a separate display screen. In some embodiments, to create the two upper images in FIG. 5, the processor 32 uses the current, tracked position of the tool 22 to select transverse and sagittal slices that contain the current location of the tool 22 from the previously-acquired CT images. In some embodiments, the processor 32 superimposes an icon or virtual image 140 on the images corresponding to the current tool location and trajectory, along with a guide line 141 showing the trajectory of the tool and/or the direction and depth to be drilled or cut. In some embodiments, to create the lower 3D view in FIG. 5, the processor 32 can use the tracked position of the tool 22 to superimpose a 3D icon 142 on the spine. The area that is to be cut from the vertebrae is also marked on the image, for example by an outline 144.

FIG. 6 is a CT image that can be used by a surgeon in the process of planning an osteotomy in which a part of one of the vertebrae is to be removed in an open surgical procedure, in accordance with another embodiment of the disclosure. As in the preceding embodiment, the surgeon has marked a relevant area 150 of the bone that is to be removed, and the processor 32 uses the marked area in identifying the corresponding 3D volume of the bone.

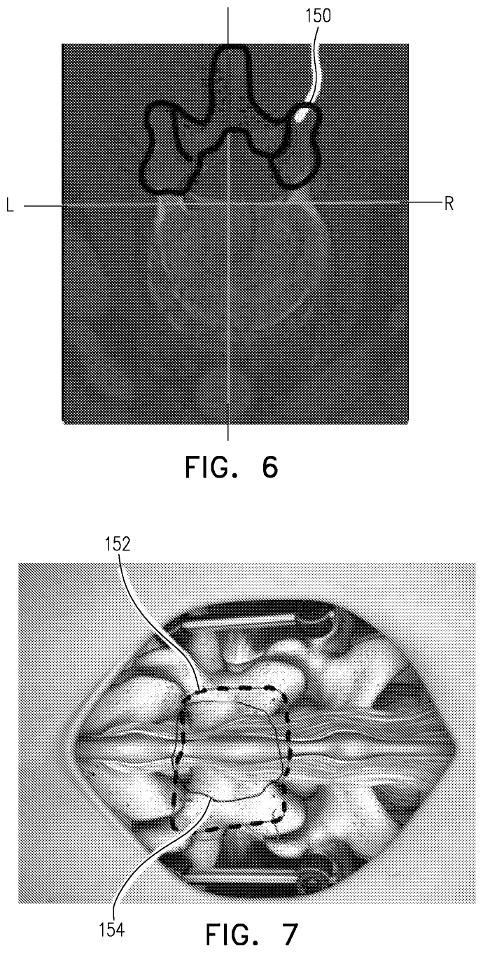

FIG. 7 is a schematic pictorial view of the part of the patient's back that is viewed by the surgeon during the osteotomy planned in FIG. 6, in accordance with an embodiment of the disclosure. In some embodiments, based on the 3D volume derived from the surgical plan, the processor 32 computes and displays an outline 152 of the area of the bone that is to be removed, as a guide for the surgeon. Outline 152 is presented, for example, on one or both of the displays 30, so that the surgeon sees the outline superimposed on the actual bone that is to be cut. In some embodiments, in the course of the operation and/or at the conclusion of the operation, the processor 32 can compute the actual volume of bone that has been removed, for example based on depth sensing measurements. In the example shown in FIG. 7, the volume of the bone that has been removed is represented by an outline 154, for comparison with outline 152.

In another embodiment, deep learning techniques are used to enable automatic or semi-automatic planning of surgical procedures based on a 3D scan of the patient's spine, such as a CT scan or depth image, utilizing technologies such as structured light, as described above. The planning is generated using convolutional neural networks (CNNs) designed for performing image segmentation. A separate CNN can be trained for each clinically-distinguished type of procedure. for example discectomy, laminectomy or vertebrectomy, and for each clinically-distinguished area of the spine, such as the cervical spine, thoracic spine, or lumbar spine.

To train the CNNs, the spine is segmented in a training set of 3D scans to facilitate localization. In general, the vertebrae are segmented. Segmentation of additional parts of the spine, such as discs or lamina, may be performed depending on the relevant clinical procedure. The input segmented 3D scans with indications of the bone and/or disc volume to be removed are used as the training set. The bone-cut volume indications are used as ground truth and may be indicated in the scans as a mask. A number of techniques can be used to obtain the bone-cut volume indications:
Using planning or labeling performed on a 3D scan captured prior to the bone and/or disc removal (preoperative or intraoperative);
Using 3D scans captured prior to the bone and/or disc removal and scans captured not long after the removal. Each pair of scans (before and after) is then registered using methods such as Iterative Closest Point. Once each pair of scans has been registered, the difference, i.e., the portion of bone and/or disc removed, is identified and indicated as the volume to be removed.

Following the training stage, the CNN is able to receive as input a segmented 3D scan and to output an indication of the volume of the bone and/or disc to be removed, for example in the form of a mask. For instance, in discectomy, the breaching disc portion will be removed. The CNN can learn to identify the breaching portion. In vertebrectomy, a vertebra with a tumor may be removed. The network may learn to identify a diseased vertebra.

Although the drawings and embodiments described above relate specifically to surgery on the spine, the principles of the present disclosure may similarly be applied in other sorts of surgical procedures, such as operations performed on the cranium and various joints, as well as dental surgery. It will thus be appreciated that the embodiments described above are cited by way of example, and that the present disclosure is not limited to what has been particularly shown and described hereinabove. Rather, the scope of the present disclosure includes both combinations and subcombinations of the various features described hereinabove, as well as variations and modifications thereof which would occur to persons skilled in the art upon reading the foregoing description and which are not disclosed in the prior art.

Indeed, although the systems and processes have been disclosed in the context of certain embodiments and examples, it will be understood by those skilled in the art that the various embodiments of the systems and processes extend beyond the specifically disclosed embodiments to other alternative embodiments and/or uses of the systems and processes and obvious modifications and equivalents thereof. In addition, while several variations of the embodiments of the systems and processes have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosed systems and processes. Any methods disclosed herein need not be performed in the order recited. Thus, it is intended that the scope of the systems and processes herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure. Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

Each of the processes, methods, and algorithms described in the preceding sections may be embodied in, and fully or partially automated by, code modules executed by one or more computer systems or computer processors comprising computer hardware. The code modules may be stored on any type of non-transitory computer-readable medium or computer storage device. such as hard drives, solid state memory, optical disc, and/or the like. The systems and modules may also be transmitted as generated data signals (for example, as part of a carrier wave or other analog or digital propagated signal) on a variety of computer-readable transmission mediums, including wireless-based and wired/cable-based mediums, and may take a variety of forms (for example, as part of a single or multiplexed analog signal, or as multiple discrete digital packets or frames). The processes and algorithms may be implemented partially or wholly in application-specific circuitry. The results of the disclosed processes and process steps may be stored, persistently or otherwise, in any type of non-transitory computer storage such as, for example, volatile or non-volatile storage.

The various features and processes described above may be used independently of one another or may be combined in various ways. All possible combinations and sub-combinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

As used herein “generate” or “generating” may include specific algorithms for creating information based on or using other input information. Generating may include retrieving the input information such as from memory or as provided input parameters to the hardware performing the generating. Once obtained, the generating may include combining the input information. The combination may be performed through specific circuitry configured to provide an output indicating the result of the generating. The combination may be dynamically performed such as through dynamic selection of execution paths based on, for example, the input information, device operational characteristics (for example, hardware resources available, power level, power source, memory levels, network connectivity, bandwidth, and the like). Generating may also include storing the generated information in a memory location. The memory location may be identified as part of the request message that initiates the generating. In some implementations, the generating may return location information identifying where the generated information can be accessed. The location information may include a memory location, network locate, file system location, or the like.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the embodiments described herein in which elements or functions may be deleted, executed out of order from that shown or discussed, including substantially concurrently or in reverse order, depending on the functionality involved, as would be understood by those skilled in the art.

All of the methods and processes described above may be embodied in, and partially or fully automated via, software code modules executed by one or more general purpose computers. For example, the methods described herein may be performed by the processors described herein and/or any other suitable computing device. The methods may be executed on the computing devices in response to execution of software instructions or other executable code read from a tangible computer readable medium. A tangible computer readable medium is a data storage device that can store data that is readable by a computer system. Examples of computer readable mediums include read-only memory, random-access memory, other volatile or non-volatile memory devices, CD-ROMs, magnetic tape, flash drives, and optical data storage devices.

Documents incorporated by reference in the present patent application are to be considered an integral part of the application except that, to the extent that any terms are defined in these incorporated documents in a manner that conflicts with definitions made explicitly or implicitly in the present specification, only the definitions in the present specification should be considered.

It should be emphasized that many variations and modifications may be made to the above-described embodiments, the elements of which are to be understood as being among other acceptable examples. All such modifications and variations are intended to be included herein within the scope of this disclosure. The foregoing description details certain embodiments. It will be appreciated, however, that no matter how detailed the foregoing appears in text, the systems and methods can be practiced in many ways. As it is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated. While the embodiments provide various features, examples, screen displays, user interface features, and analyses, it is recognized that other embodiments may be used.

1. A system for image-guided surgery, comprising:
a near-eye unit, comprising a see-through augmented-reality display, which is configured to display graphical information with respect to a region of interest (ROI) on a body of a patient, including a bone inside the body, that is viewed through the display by a user wearing the near-eye unit; and
a processor, which is configured to access three-dimensional (3D) image data with respect to the bone, to process the 3D image data so as to identify a first 3D shape of the bone prior to a surgical procedure on the bone and a second 3D shape of the bone following the surgical procedure, to generate, based on the first and second 3D shapes, an image showing a part of the bone that was removed in the surgical procedure, and to present the image on the see-through augmented-reality display.

2. The system according to claim 1, wherein the processor is configured to access a plan of the surgical procedure and based on the plan, to present a guide for cutting the bone on the see-through augmented-reality display.

3. The system according to claim 2, wherein the processor is configured to compare the second 3D shape to the plan and to present an indication of a deviation between the part of the bone that was removed and the plan on the augmented-reality display.

4. The system according to claim 2, wherein the processor is configured to present the guide as an outline of an area of the bone that is to be removed, wherein the outline is superimposed on the bone in the see-through augmented-reality display.

5. The system according to claim 2, wherein the processor is configured to present on the see-through augmented-reality display an icon indicating a position of a tool used in cutting the bone and a line showing a trajectory that the tool is to take in cutting the bone according to the plan.

6. The system according to claim 2, wherein the processor is configured to present the guide on the see-through augmented-reality display together with an image of the bone, such that the guide and the image of the bone are overlaid on an actual location of the bone in the body.

7. The system according to claim 2, wherein the processor is configured to present the guide on the see-through augmented-reality display such that the guide is overlaid on actual bone that is to be cut in open surgery.

8. The system according to any one of claims 1-7, wherein the processor is configured to process the 3D image data at one or more times during the surgical procedure so as to identify one or more intermediate 3D shapes of the bone during the surgical procedure, and present the part of the bone removed at each of the one or more times on the see-through augmented-reality display.

9. The system according to any one of claims 1-7, wherein the near-eye unit comprises a depth sensor, which is configured to generate depth data with respect to the ROI, and wherein the processor is configured to generate the image showing the part of the bone using the depth data.

10. The system according to claim 9, wherein the processor is configured to measure and display a volume of the bone that was removed during the surgical procedure based on the depth data.

11. The system according to any one of claims 1-7, wherein the processor is configured to process the 3D image data using a convolutional neural network (CNN) so as to generate an indication of a volume of the bone to be removed in a surgical procedure.

12. The system according to any one of claims 1-7, wherein the processor is configured to access 3D tomographic data with respect to the body of the patient and to generate the image showing the part of the bone using the 3D tomographic data.

13. A method for image-guided surgery, comprising:
processing first three-dimensional (3D) image data with respect to a bone inside a body of a patient so as to identify a first 3D shape of the bone prior to a surgical procedure on the bone;
processing second 3D image data so as to identify a second 3D shape of the bone following the surgical procedure;
generating, based on the first and second 3D shapes, an image showing a part of the bone that was removed in the surgical procedure; and
presenting the image on a see-through augmented-reality display, such that the image is overlaid on a region of interest (ROI) on the body of a patient that contains the bone inside the body and is viewed through the display.

14. The method according to claim 13, and comprising accessing a plan of the surgical procedure and based on the plan, presenting a guide for cutting the bone on the see-through augmented-reality display.

15. The method according to claim 14, and comprising comparing the second 3D shape to the plan and presenting an indication of a deviation between the part of the bone that was removed and the plan on the augmented-reality display.

16. The method according to claim 14, wherein presenting the guide comprises displaying an outline of an area of the bone that is to be removed, wherein the outline is superimposed on the bone in the see-through augmented-reality display.

17. The method according to claim 14, wherein presenting the guide comprises presenting on the see-through augmented-reality display an icon indicating a position of a tool used in cutting the bone and a line showing a trajectory that the tool is to take in cutting the bone according to the plan.

18. The method according to claim 14, wherein presenting the guide comprises displaying the guide on the see-through augmented-reality display together with an image of the bone, such that the guide and the image of the bone are overlaid on an actual location of the bone in the body.

19. The method according to claim 14, wherein presenting the guide comprises displaying the guide on the see-through augmented-reality display such that the guide is overlaid on actual bone that is to be cut in open surgery.

20. The method according to any one of claims 13-19, and comprising acquiring and processing further 3D image data at one or more times during the surgical procedure so as to identify one or more intermediate 3D shapes of the bone during the surgical procedure, and presenting the part of the bone removed at each of the one or more times on the see-through augmented-reality display.

21. The method according to any one of claims 13-19, wherein the 3D image data comprise depth data, which are acquired with respect to the ROI by a depth sensor, and wherein processing the first and second 3D image data comprises generating the image showing the part of the bone using the depth data.

22. The method according to claim 21, and comprising measuring and displaying a volume of the bone that was removed during the surgical procedure based on the depth data.

23. The method according to any one of claims 13-19, and comprising processing the first 3D image data using a convolutional neural network (CNN) so as to generate an indication of a volume of the bone to be removed in a surgical procedure.

24. The method according to any one of claims 13-19, wherein processing the first 3D image data comprises accessing 3D tomographic data with respect to the body of the patient and generating the image showing the part of the bone using the 3D tomographic data.

25. A computer software product, comprising a tangible, non-transitory computer-readable medium in which program instructions are stored, which instructions, when read by a computer, cause the computer to process first three-dimensional (3D) image data with respect to a bone inside a body of a patient so as to identify a first 3D shape of the bone prior to a surgical procedure on the bone, to process second 3D image data so as to identify a second 3D shape of the bone following the surgical procedure, to generate, based on the first and second 3D shapes, an image showing a part of the bone that was removed in the surgical procedure, and to present the image on a see-through augmented-reality display, such that the image is overlaid on a region of interest (ROI) on the body of a patient that contains the bone inside the body and is viewed through the display.

26. A system for image-guided surgery comprising:
a see-through augmented reality display configured to display a bone with respect to a region of interest (ROI) on a body of a patient, the bone being disposed inside the patient; and
a processor and a memory for storing instructions that, when executed by the processor cause the system to:
access three-dimensional (3D) image data related to the bone;
determine a first 3D shape of the bone prior to removing a portion of the bone;
determine a second 3D shape of the bone after the portion is removed;
generate an image of the portion of the bone removed; and
displaying the image on the see-through augmented-reality display so as to be viewable by the user in the ROI on the body of the patient.

27. A method for image-guided surgery comprising:
determining a first three-dimensional (3D) shape of a bone prior to removing a portion of the bone, the bone being disposed inside a patient;
determining a second 3D shape of the bone after the portion is removed;
generating, based on the first and second 3D shapes, an image of the portion of the bone removed; and
displaying the image on a see-through augmented-reality display so as to be viewable by a user in a region of interest (ROI) on the body of the patient.

28. The method according to claim 27, further comprising:
displaying a plan for the removal of the portion of the bone on the see-through angmented-reality display, the plan illustrating the bone after one or more cuts to the bone; and
if there is a deviation between the second 3D shape and the plan, displaying an indication of the deviation on the see-through angmented-reality display.
